(19) United States
(12) Patent Application Publication
(10) Pub. No.: US 2016/0113676 A1
(43) Pub. Date: Apr. 28, 2016

(54) THROMBUS ASPIRATION CATHETER AND METHOD OF USING THE SAME

(71) Applicant: TERUMO KABUSHIKI KAISHA, Tokyo (JP)

(72) Inventors: Yuichi TADA, Tokyo (JP); Masahiro AKIYAMA, Fujinomiya-shi (JP); Yutaka TANO, Fujinomiya-shi (JP); Naoko TANAKA, Tokyo (JP); Takehiro URA, Yokohama-shi (JP); Yuji ONIMURA, Fujinomiya-shi (JP)

(73) Assignee: TERUMO KABUSHIKI KAISHA, Tokyo (JP)

(21) Appl. No.: 14/924,155

(22) Filed: Oct. 27, 2015

(30) Foreign Application Priority Data
Oct. 28, 2014 (JP) 2014-219559

(51) Int. Cl. A61B 17/3207; A61B 17/22

(52) U.S. Cl. CPC A61B 17/320783; A61B 17/22; A61B 2017/320791; A61B 2017/22001; A61B 2017/22079

(57) ABSTRACT

A thrombus aspiration catheter is disclosed which can shear a thrombus and efficiently aspirate the thrombus. When the pressure of the inside of an inner tube is made negative, an outer tube is rotated around an axis relative to the inner tube, and an inner opening portion and an outer opening portion are positioned at an open position at which the inner opening portion and the outer opening portion overlap each other, a thrombus enters the inside of the inner tube through the inner opening portion and the outer opening portion. When the outer tube is further rotated up to a closed position at which the whole outer opening portion does not overlap the inner opening portion, the thrombus is sheared by an edge portion of the inner opening portion and an edge portion of the outer opening portion and into the inside of the inner tube.

### CROSS-REFERENCES TO RELATED APPLICATIONS

This application claims priority to Japanese Patent Application No. 2014-219559 filed on Oct. 28, 2014, the entire content of which is incorporated herein by reference.

### TECHNICAL FIELD

The present disclosure relates to a thrombus aspiration catheter and a method of using the same, and particularly to a catheter for aspirating a thrombus by cutting the thrombus from the inside of a blood vessel.

### BACKGROUND DISCUSSION

It has been known that a thrombus generated in a blood vessel such as a coronary artery in the heart is aspirated and removed using a thrombus aspiration catheter.

As this type of a thrombus aspiration catheter, for example, a catheter in which a plurality of aspiration ports are formed on a side wall of a thrombus aspiration lumen and which captures a thrombus from the plurality of aspiration ports into the thrombus aspiration lumen by making the pressure of the inside of the thrombus aspiration lumen negative, and removes the captured thrombus from the body, is disclosed in JP-A-2008-55102.

In addition, JP-T-2012-509733 discloses that a conveyer screw is incorporated in an operation head which is disposed at a distal end of a catheter and an opening is formed in a side portion of the operation head, a thrombus is sheared through an interaction between the conveyer screw and the opening by rotating the conveyer screw, and then, thrombi are continuously drawn in using the conveyer screw.

However, since the thrombus aspiration catheter in JP-A-2008-55102 has no function of shearing a thrombus, it can be difficult to capture a thrombus having a larger size than that of the aspiration ports, or since the plurality of aspiration ports are opened at all times, the aspiration force decreases even if the pressure of the inside of the thrombus aspiration lumen is made to be negative. Therefore, there is a concern that it may be impossible to efficiently aspirate and remove a captured thrombus.

In contrast, in the catheter in JP-T-2012-509733, since the conveyer screw incorporated in the operation head is rotated, there is a concern that the structure may become complicated and the size of the distal end of the catheter may become large.

### SUMMARY

A thrombus aspiration catheter is disclosed, which can shear a thrombus and can efficiently aspirate the thrombus while having a simple structure.

According to the disclosure, a thrombus aspiration catheter is disclosed for aspirating a thrombus by being inserted into a blood vessel and cutting the thrombus, including an inner tube in which an inner opening portion is formed in the vicinity of a distal portion thereof, and an outer tube in which an outer opening portion is formed in the vicinity of a distal portion thereof and which is positioned outside the inner tube so as to cover the inner tube so as to be rotatable relative to the inner tube between an open position at which at least a part of the outer opening portion overlaps the inner opening portion and a closed position at which the whole outer opening portion does not overlap the inner opening portion. When the outer tube is at the open position, the thrombus enters the inside of the inner tube through the outer opening portion and the inner opening portion, the thrombus which has entered the inside of the inner tube is sheared by both edge portions of the outer opening portion and the inner opening portion by relatively moving the outer tube to the closed position, and the thrombus is aspirated through the inside of the inner tube.

In accordance with an exemplary embodiment, it can be preferable the outer tube is disposed so as to be rotatable around an axis of the inner tube or is disposed so as to be slidable in an axial direction of the inner tube.

The thrombus aspiration catheter can be configured such that the inner tube has a plurality of the inner opening portions and the outer tube has a plurality of the outer opening portions respectively corresponding to the plurality of inner opening portions.

A method is disclosed of using a thrombus aspiration catheter, including: inserting the aforesaid thrombus aspiration catheter into a blood vessel; making the pressure of the inside of the inner tube negative; making a thrombus enter the inside of the inner tube through the outer opening portion and the inner opening portion by positioning the outer tube at the open position; and shearing the thrombus which has entered the inside of the inner tube using both edge portions of the outer opening portion and the inner opening portion by relatively moving the outer tube to the closed position, and aspirating the thrombus through the inside of the inner tube.

According to this disclosure, a thrombus is sheared by both edge portions of an outer opening portion of an outer tube and an inner opening portion of an inner tube by relatively moving the outer tube which is disposed so as to be movable relative to the inner tube from an open position to a closed position, and the thrombus is aspirated through the inside of the inner tube. Therefore, the thrombus aspiration catheter can shear a thrombus and can efficiently aspirate while having a simple structure.

A thrombus aspiration catheter is disclosed for aspirating a thrombus by being inserted into a blood vessel and cutting the thrombus, comprising: an inner tube having a plurality of inner opening portions in a distal portion of the inner tube; and an outer tube having a plurality of outer opening portions in a distal portion of the outer tube, each of the plurality of inner opening portions respectively corresponding to the plurality of outer opening portions, and wherein the outer tube is positioned outside the inner tube so as to cover the inner tube so as to be rotatable relative to the inner tube between an open position at which at least a part of the plurality of outer opening portions overlaps the plurality of inner opening portions and a closed position at which an entirety of the plurality of outer opening portions does not overlap the plurality of inner opening portions.

### BRIEF DESCRIPTION OF THE DRAWINGS

FIG. 1 is a side view showing an overall structure of a thrombus aspiration catheter according to an exemplary embodiment of the disclosure.

FIG. 2 is a partial side view showing a structure in the vicinity of a distal end of the thrombus aspiration catheter of the exemplary embodiment as shown in FIG. 1.

FIG. 3A is a sectional front view showing a structure in the vicinity of the distal end of the thrombus aspiration catheter when an outer tube is at an open position.

FIG. 3B is a sectional front view showing a structure in the vicinity of the distal end of the thrombus aspiration catheter when the outer tube is at a closed position.

FIGS. 4A-4C are sectional front views showing a method of using the thrombus aspiration catheter according to an exemplary embodiment in stages.

FIG. 5 is a partial sectional front view showing a structure in the vicinity of an outer opening portion and an inner opening portion of a thrombus aspiration catheter according to a modification example of the exemplary embodiment as shown in FIG. 1.

FIG. 6A is a sectional front view showing a structure in the vicinity of a distal end of a thrombus aspiration catheter when an outer tube is at an open position.

FIG. 6B is a sectional front view showing a structure in the vicinity of a distal end of a thrombus aspiration catheter when the outer tube is at a closed position.

FIG. 7 is a partial side view showing a state in the vicinity of a distal end of a thrombus aspiration catheter according to an exemplary embodiment when an outer tube is at an open position.

FIG. 8 is a partial side view showing a state in the vicinity of the distal end of the thrombus aspiration catheter according to the exemplary embodiment as shown in FIG. 7 when the outer tube is at a closed position.

FIG. 9 is a partial sectional side view showing a structure in the vicinity of an outer opening portion and an inner opening portion of a thrombus aspiration catheter according to a modification example of the exemplary embodiment as shown in FIG. 7.

FIG. 10 is a partial sectional side view showing a structure in the vicinity of a distal end of an outer tube of a thrombus aspiration catheter according to another modification example of the exemplary embodiment as shown in FIG. 7.

FIG. 11 is a partial side view showing a structure in the vicinity of a distal end of a thrombus aspiration catheter according to still another modification example of the exemplary embodiment as shown in FIG. 7.

FIG. 12 is a partial perspective view showing a structure in the vicinity of a distal end of an outer tube of a thrombus aspiration catheter according to an exemplary embodiment.

FIG. 13 is a partial side view showing the vicinity of a proximal end of a thrombus aspiration catheter according to an exemplary embodiment.

### DETAILED DESCRIPTION

Hereinafter, examples of the disclosure will be described based on the accompanying drawings.

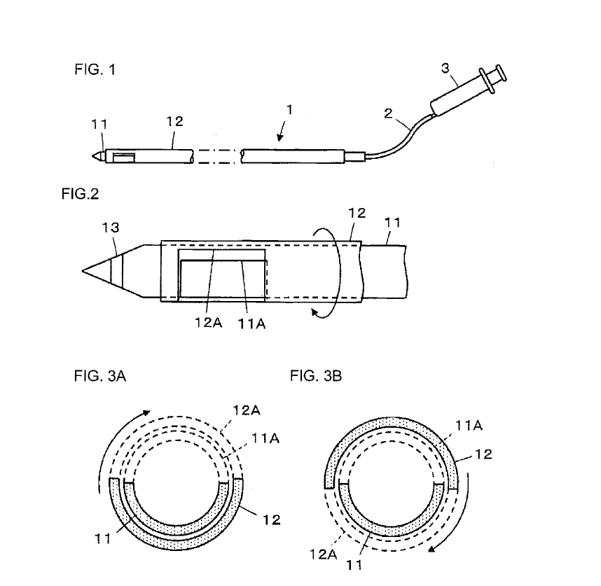

The appearance of a thrombus aspiration catheter 1 according to an exemplary embodiment is shown in FIG. 1. The thrombus aspiration catheter 1 has a double tube structure consisting of an elongated inner tube 11, and an elongated outer tube 12, which is disposed outside the inner tube 11 so as to cover the inner tube 11. Each of a distal end and a proximal end of the outer tube 12 is open, the outer tube is disposed so as to be rotatable around the axis of the inner tube 11, and the inner tube 11 is exposed from the distal end and the proximal end of the outer tube 12.

A syringe 3 for making the pressure of the inside of the inner tube 11 negative can be connected to the proximal end of the inner tube 11 through a connecting tube 2.

As shown in FIG. 2, one rectangular inner opening portion 11A is formed in a side wall portion in the vicinity of a distal portion of the inner tube 11 so as to allow communication between the inside and the outside of the inner tube 11. Similarly, one rectangular outer opening portion 12A is formed in a side wall portion in the vicinity of a distal portion of the outer tube 12 so as to allow communication between the inside and the outside of the outer tube 12.

The inner opening portion 11A and the outer opening portion 12A are formed at a position of the same axial direction as each other, are almost the same size as each other, and are configured such that the overlapping state of the inner opening portion 11A and the outer opening portion 12A changes in accordance with the rotational position of the outer tube 12 by rotating the outer tube 12 around the axis of the inner tube 11.

For example, the outer tube 12 is disposed so as to be rotatable between an open position at which the inner opening portion 11A and the outer opening portion 12A overlap each other as shown in FIG. 3A and a closed position at which the whole outer opening portion 12A does not overlap the inner opening portion 11A as shown in FIG. 3B. When the outer tube 12 is at the open position, the inside of the inner tube 11 communicates with the outside of the outer tube 12 through the inner opening portion 11A and the outer opening portion 12A, and when the outer tube 12 is at the closed position, the communication between the inside of the inner tube 11 and the outside of the outer tube 12 is blocked and the inside of the inner tube 11 is isolated from the outside of the outer tube 12.

Note that, as shown in FIG. 2, a contrast marker 13 can be disposed at a distal portion of the inner tube 11, which is exposed to the front side from the distal end of the outer tube 12. The contrast marker 13 can be used for grasping the distal position of the thrombus aspiration catheter 1, which has been inserted into a blood vessel, from the outside of a living body through X-ray contrast.

In addition, a guide wire insertion port which is used for passing a guide wire therethrough and not shown in the drawing is formed at a distal portion of the inner tube 11, and is connected to the inside of the inner tube 11. Alternately, a lumen for guide wire is set to be formed in the inner tube 11 along the longitudinal direction of the inner tube 11.

Next, a method of using the thrombus aspiration catheter 1 according to an exemplary embodiment will be described.

First, a guide wire not shown in the drawing is inserted into a blood vessel and is advanced up to a thrombus-generated site. Then, the guide wire is passed through the guide wire insertion port at the distal portion of the inner tube 11 or the lumen for guide wire and the thrombus aspiration catheter 1 is inserted into the thrombus-generated site in the blood vessel having the guide wire as a leading guide. At this time, the distal position of the thrombus aspiration catheter 1 in the blood vessel is confirmed by recognizing the contrast marker 13 by performing X-ray contrast.

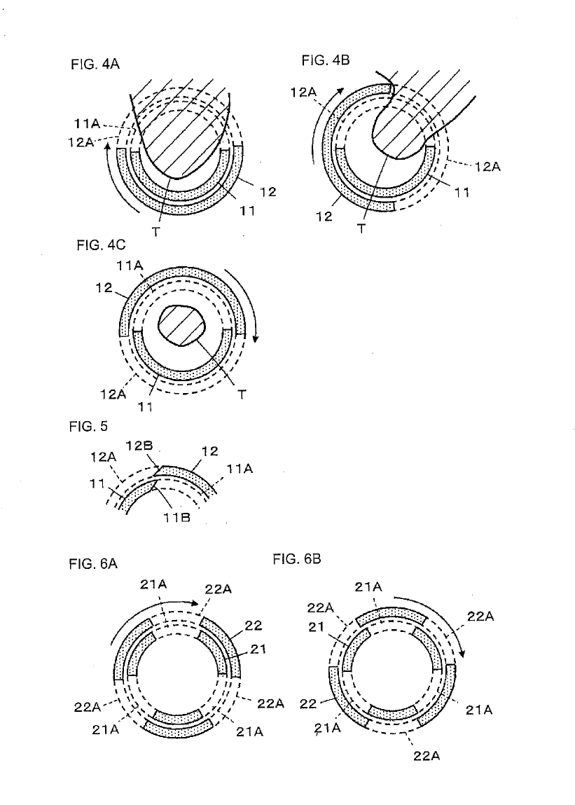

Next, the pressure of the inside of the inner tube 11 is made negative using the syringe 3, which has been connected to the proximal end of the inner tube 11 through the connecting tube 2, the outer tube 12 is rotated around the axis relative to the inner tube 11, and the inner opening portion 11A and the outer opening portion 12A are positioned at an open position at which the inner opening portion and the outer opening portion overlap each other as shown in FIG. 4A. Accordingly, the inside of the inner tube 11 is connected to the outside of the outer tube 12, and a thrombus T in a blood vessel enters a state in which the thrombus enters the inside the inner tube 11 through the inner opening portion 11A and the outer opening portion 12A.

If the region in which the inner opening portion 11A and the outer opening portion 12A overlap each other is narrowed by relatively rotating the outer tube 12 as shown in FIG. 4B, the thrombus T is interposed between an edge portion of the inner opening portion 11A and an edge portion of the outer opening portion 12A. Then, as shown in FIG. 4C, when the outer tube 12 is further rotated up to the closed position at which the whole outer opening portion 12A does not overlap the inner opening portion 11A, the thrombus T is sheared by the edge portion of the inner opening portion 11A and the edge portion of the outer opening portion 12A and is taken into the inner tube 11.

At this time, the outer tube 12 is at the closed position to enter a state in which the communication between the inside of the inner tube 11 and the outside of the outer tube 12 is blocked. Therefore, the negative pressure in the inner tube 11 increases and the thrombus T, which has been taken into the inner tube 11, is aspirated to a proximal end of the inner tube 11.

A next thrombus T is taken into the inner tube 11 in the same manner as the above by continuously rotating the outer tube 12, is sheared, and is aspirated to the proximal end of the inner tube 11.

The thrombus aspiration catheter can shear a thrombus T generated in a blood vessel and can efficiently aspirate and remove the thrombus while having a simple structure in which the inner opening portion 11A and the outer opening portion 12A are respectively formed in the vicinity of the distal portions of the inner tube 11 and the outer tube 12 in this manner.

In addition, since the inner opening portion 11A and the outer opening portion 12A are respectively formed in the vicinity of the distal portions of the inner tube 11 and the outer tube 12, the reduction in the size of the thrombus aspiration catheter can be achieved.

In accordance with an exemplary embodiment, a material having flexibility to some extent is preferable as a material forming the inner tube 11 and the outer tube 12, and examples thereof include polymeric materials such as polyolefins (for example, polyethylene, polypropylene, polybutene, an ethylene-propylene copolymer, an ethylene-vinyl acetate copolymer, an ionomer, a mixture of two or more thereof or the like), polyvinyl chloride, polyamide, a polyamide elastomer, polyester, a polyester elastomer, polyurethane, a polyurethane elastomer, polyimide, fluoro resin or the like or a mixture thereof, or two or more kinds of the aforesaid polymeric materials.

In addition, the contrast marker 13 can be formed of a material which can easily be recognized through X-ray contrast, and examples thereof include gold, platinum, iridium, tungsten, or alloys thereof, or silver-palladium alloys, for example.

Note that, in FIGS. 3A and 4A, the rotational position of the outer tube 12 at which almost the whole region of the outer opening portion 12A overlaps with the inner opening portion 11A has been called an open position. However, a thrombus T can be taken into the inner tube 11 as long as at least a part of the outer opening portion 12A overlaps the inner opening portion 11A and such a rotational position of the outer tube 12 can also be called the open position.

As shown in FIG. 5, a cutting portion 11B which is pointed toward a circumferential direction of the inner tube 11 can be formed at the edge portion of the inner opening portion 11A of the inner tube 11 and a cutting portion 12B which is pointed toward a circumferential direction of the outer tube 12 can be formed at the edge portion of the outer opening portion 12A of the outer tube 12, to configure the thrombus aspiration catheter so as to shear the thrombus T using these cutting portions 11B and 12B.

In the aforesaid exemplary embodiment, the inner tube 11 and the outer tube 12 have one inner opening portion 11A and one outer opening portion 12A which respectively correspond thereto, but the present disclosure is not limited thereto. For example, as shown in FIG. 6A, the inner tube 21 may have three inner opening portions 21A which are disposed at even intervals in the circumferential direction and the outer tube 22 may have three outer opening portions 22A which are disposed at even intervals in the circumferential direction corresponding to the three inner opening portions 21A of the inner tube 21.

The outer tube 22 is rotated between an open position at which the inner opening portions 21A and the outer opening portions 22A corresponding to the inner opening portions respectively overlap each other as shown in FIG. 6A and a closed position at which all of the outer opening portions 22A do not overlap any inner opening portions 21A as shown in FIG. 6B, by rotating the outer tube 22 around the axis relatively to the inner tube 21.

Even with such a configuration, similarly to the exemplary embodiment as disclosed above, the thrombus T generated in a blood vessel can be sheared and efficiently aspirated and removed.

Similarly, the inner tube 21 may have two or four or more inner opening portions 21A which are disposed at even intervals in the circumferential direction and the outer tube 22 may have two or four or more outer opening portions 22A which are disposed at even intervals in the circumferential direction corresponding to the inner opening portions 21A of the inner tube 21.

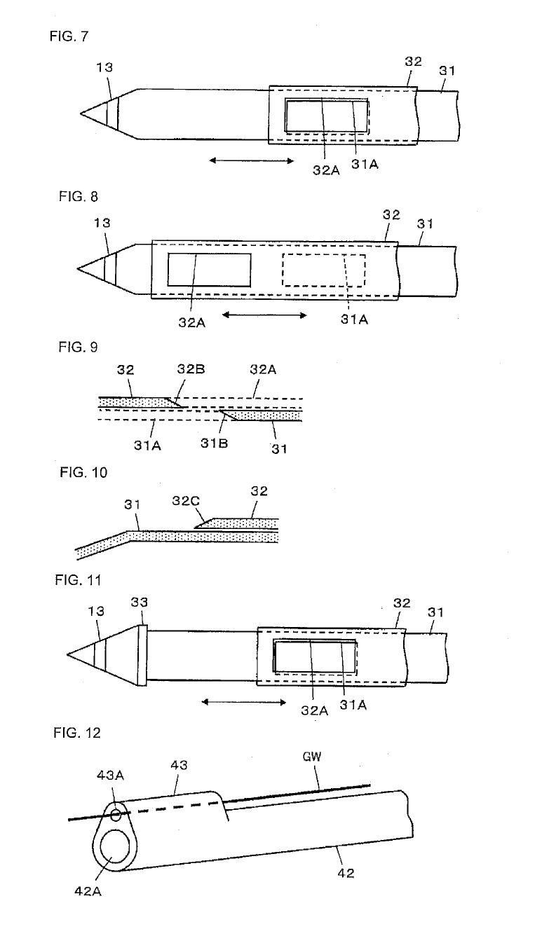

A structure in the vicinity of a distal end of a thrombus aspiration catheter according to an exemplary embodiment is shown in FIG. 7.

In the thrombus aspiration catheter according to the exemplary embodiment shown in FIG. 7, an outer tube 32 is disposed so as to be slidable in an axial direction of an inner tube 31. One rectangular inner opening portion 31A which allows communication between the inside and the outside of the inner tube 31 is formed in a side wall portion in the vicinity of a distal portion of the inner tube 31 and one rectangular outer opening portion 32A which allows communication between the inside and the outside of the outer tube 32 is formed in a side wall portion in the vicinity of a distal portion of the outer tube 32.

The inner opening portion 31A and the outer opening portion 32A are formed at a position of the same circumferential direction as each other, are almost the same size as each other, and are configured such that the overlapping state of the inner opening portion 31A and the outer opening portion 32A changes in accordance with the sliding position of the outer tube 32 by making the outer tube 32 slide in the axial direction relatively to the inner tube 31.

For example, the outer tube 32 is disposed so as to be relatively slidable between an open position at which the inner opening portion 31A and the outer opening portion 32A overlap each other as shown in FIG. 7 and a closed position at which the whole outer opening portion 32A does not overlap the inner opening portion 31A as shown in FIG. 8. When the outer tube 32 is at the open position, the inside of the inner tube 31 communicates with the outside of the outer tube 32 through the inner opening portion 31A and the outer opening portion 32A, and when the outer tube 32 is at the closed position, the communication between the inside of the inner tube 31 and the outside of the outer tube 32 is blocked and the inside of the inner tube 31 is isolated from the outside of the outer tube 32.

With the configuration in which the outer tube 32 is made to slide in the axial direction relative to the inner tube 31 in this manner, a thrombus T in a blood vessel enters the inside of the inner tube 31 through the inner opening portion 31A and the outer opening portion 32A by positioning the outer tube 32 at the open position, and when the outer tube 32 is made to slide up to the closed position, the thrombus T is sheared by an edge portion of the inner opening portion 31A and an edge portion of the outer opening portion 32A, is taken into the inside of the inner tube 31, and is aspirated to a proximal end of the inner tube 11 through the inside of the inner tube 11 in which the negative pressure has increased.

That is, similarly to exemplary embodiment as disclosed above, the thrombus T generated in a blood vessel can be sheared and efficiently aspirated and removed.

In accordance with an exemplary embodiment, as shown in FIGS. 7 and 8, a contrast marker 13 can be disposed at a distal portion of the inner tube 31, which is exposed to the front side from the distal end of the outer tube 32, similarly to the contrast marker used in the exemplary embodiment as disclosed above.

As shown in FIG. 9, a cutting portion 31B which is pointed toward an axial direction of the inner tube 31 can be formed at the edge portion of the inner opening portion 31A of the inner tube 31 and a cutting portion 32B which is pointed toward an axial direction of the outer tube 32 can be formed at the edge portion of the outer opening portion 32A of the outer tube 32, to configure the thrombus aspiration catheter so as to shear the thrombus T using these cutting portions 31B and 32B.

In addition, as shown in FIG. 10, a cutting portion 32C which is pointed forward along the axial direction of the outer tube 32 may be formed at the distal portion of the outer tube 32 to configure the thrombus aspiration catheter so as to peel off the thrombus T in a blood vessel using the cutting portion 32C when making the outer tube 32 slide on the inner tube 31.

Furthermore, if an annular stopper 33 having a larger diameter than that of the outer tube 32 is formed at the distal portion of the inner tube 31 which is exposed forward from the distal end of the outer tube 32 as shown in FIG. 11, the outer tube 32 is prevented from protruding forward from the inner tube 31.

In accordance with an exemplary embodiment, the thrombus aspiration catheter may be configured such that the inner tube 31 has a plurality of inner opening portions 31A which are disposed at even intervals in the circumferential direction and the outer tube 32 has a plurality of outer opening portions 32A which are disposed at even intervals in the circumferential direction corresponding to the plurality of inner opening portions 31A of the inner tube 31.

In addition, even if the sliding operation and the rotating operation are simultaneously performed by rotating the outer tube 32 around the axis relative to the inner tube 31 as well as making the outer tube 32 slide in the axial direction relative to the inner tube 31, the thrombus T generated in a blood vessel can be sheared and efficiently aspirated and removed as long as the outer tube 32 relatively moves between the open position at which the inner opening portion 31A and the outer opening portion 32A overlap each other and the closed position at which the whole outer opening portion 32A does not overlap the inner opening portion 31A.

In the aforesaid exemplary embodiments, the outer tube 12, 22, or 32 is disposed so as to be movable with respect to the inner tube 11, 21, or 31. However, both sides may be relatively moved, or the inner tube 11, 21, or 31 may be disposed so as to be movable with respect to the outer tube 12, 22, or 32.

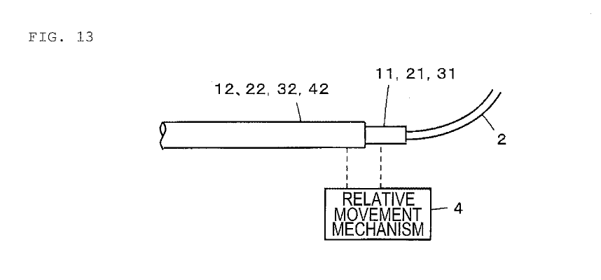

In the aforesaid exemplary embodiments, an outer tube 42 having a double lumen structure as shown in FIG. 12 can be used instead of the outer tube 12, 22, or 32.

An inner tube insertion lumen 42A is formed in the outer tube 42 over the entire length of the outer tube from a proximal portion to a distal portion thereof. In addition, a guide wire insertion portion 43 is protrusively formed in an outside portion at a distal end of the outer tube 42 and a guide wire insertion lumen 43A is formed in this guide wire insertion portion 43.

The outer opening portion 12A, 22A, or 32A which is shown in the exemplary embodiment is formed in the vicinity of a distal portion of the outer tube 42 and the inner tube 11, 21, or 31 is inserted into the inner tube insertion lumen 42A.

A guide wire GW is inserted into a blood vessel and is advanced up to a thrombus-generated site. Then, the guide wire GW is passed through the guide wire insertion lumen 43A and the thrombus aspiration catheter is inserted into the thrombus-generated site in the blood vessel having the guide wire GW as a leading guide. A thrombus T can be aspirated and removed by shearing the thrombus similarly to the exemplary embodiments by moving the outer tube 42 relative to the inner tube 11, 21, or 31.

At this time, since the guide wire GW is passed through the guide wire insertion lumen 43A, the outer tube 42 can slide along the guide wire GW only in an axial direction without being rotated around the axis, and the inner tube 11, 21, or 31 which is inserted into the inner tube insertion lumen 42A can be rotated around the axis and slide in the axial direction. Accordingly, in a case where the inner tube 11 or 21 is inserted into the inner tube insertion lumen 42A, the thrombus T can be sheared by rotating the inner tube 11 or 21 with respect to the outer tube 42.

The winding of the guide wire GW to the outer tube 42 is avoided using the outer tube 42, which can slide only in the axial direction along the guide wire GW in this manner.

In the aforesaid exemplary embodiments, relative movement between the inner tube 11, 21, or 31 and the outer tube 12, 22, 32, or 42 can be manually performed at the proximal portion of the thrombus aspiration catheter by an operator. For example, as shown in FIG. 13, the thrombus aspiration catheter can be configured such that a relative movement mechanism 4, such as an electric motor, an electromagnetic cylinder, an air cylinder or the like, which has an actuator is connected to the proximal portion of the inner tube 11, 21, or 31 and the proximal portion of the outer tube 12, 22, 32, or 42 and the relative movement is performed by this relative movement mechanism 4.

All of the inner opening portions 11A, 21A, and 31A shown in the exemplary embodiments and all of the outer opening portions 12A, 22A, and 32A shown in the exemplary embodiments are formed in a rectangular shape. However, the rectangular shape is merely an example, and the present disclosure is not limited to the shape of these inner opening portions and outer opening portions.

Furthermore, the syringe 3 can be used in order to make the pressure of the inside of the inner tube 11, 21, or 31 negative. However, an aspiration device, which can continuously generate negative pressure, can be used instead of the syringe 3.

In addition, this disclosure can also be applied to any of a so-called over-the-wire type catheter, into which a guide wire is inserted from a proximal portion to a distal portion of the catheter, and a so-called rapid exchange type catheter, through which a guide wire is passed only at a distal portion.

The detailed description above describes a thrombus aspiration catheter and a method of using the same. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A thrombus aspiration catheter for aspirating a thrombus by being inserted into a blood vessel and cutting the thrombus, comprising:
an inner tube in which an inner opening portion is formed in a distal portion of the inner tube; and
an outer tube in which an outer opening portion is formed in a distal portion of the outer tube and which is positioned outside the inner tube so as to cover the inner tube so as to be rotatable relative to the inner tube between an open position at which at least a part of the outer opening portion overlaps the inner opening portion and a closed position at which a whole outer opening portion does not overlap the inner opening portion,
wherein when the outer tube is at the open position, the thrombus enters an inside of the inner tube through the outer opening portion and the inner opening portion, the thrombus which has entered the inside of the inner tube is sheared by both edge portions of the outer opening portion and the inner opening portion by relatively moving the outer tube to the closed position, and the thrombus is aspirated through the inside of the inner tube.

2. The thrombus aspiration catheter according to claim 1,
wherein the outer tube is configured so as to be rotatable around an axis of the inner tube.

3. The thrombus aspiration catheter according to claim 1,
wherein the outer tube is configured so as to be slidable in an axial direction of the inner tube.

4. The thrombus aspiration catheter according to claim 1,
wherein the inner tube has a plurality of inner opening portions, and
wherein the outer tube has a plurality of outer opening portions respectively corresponding to the plurality of inner opening portions.

5. The thrombus aspiration catheter according to claim 2,
wherein the inner tube has a plurality of inner opening portions, and
wherein the outer tube has a plurality of outer opening portions respectively corresponding to the plurality of inner opening portions.

6. The thrombus aspiration catheter according to claim 3,
wherein the inner tube has a plurality of inner opening portions, and
wherein the outer tube has a plurality of outer opening portions respectively corresponding to the plurality of inner opening portions.

7. A thrombus aspiration catheter for aspirating a thrombus by being inserted into a blood vessel and cutting the thrombus, comprising:
an inner tube having a plurality of inner opening portions in a distal portion of the inner tube; and
an outer tube having a plurality of outer opening portions in a distal portion of the outer tube, each of the plurality of inner opening portions respectively corresponding to the plurality of outer opening portions, and wherein the outer tube is positioned outside the inner tube so as to cover the inner tube so as to be rotatable relative to the inner tube between an open position at which at least a part of the plurality of outer opening portions overlaps the plurality of inner opening portions and a closed position at which an entirety of the plurality of outer opening portions does not overlap the plurality of inner opening portions.

8. The thrombus aspiration catheter according to claim 7, wherein when the outer tube is at the open position, the thrombus enters an inside of the inner tube through an outer opening portion and an inner opening portion of the plurality of outer opening portions and the plurality of inner opening portions, and wherein the thrombus which has entered the inside of the inner tube is sheared by both edge portions of the outer opening portion and the inner opening portion by relatively moving the outer tube to the closed position.

9. The thrombus aspiration catheter according to claim 8, wherein the thrombus is aspirated through the inside of the inner tube.

10. The thrombus aspiration catheter according to claim 7,
wherein the outer tube is configured so as to be rotatable around an axis of the inner tube.

11. The thrombus aspiration catheter according to claim 7,
wherein the outer tube is configured so as to be slidable in an axial direction of the inner tube.

12. A method of using a thrombus aspiration catheter, the method comprising:
inserting the thrombus aspiration catheter into a blood vessel, the thrombus aspiration catheter including an inner tube in which an inner opening portion is formed in a distal portion of the inner tube, and an outer tube in which an outer opening portion is formed in a distal portion of the outer tube and which is positioned outside the inner tube so as to cover the inner tube so as to be rotatable relative to the inner tube between an open position at which at least a part of the outer opening portion overlaps the inner opening portion and a closed position at which a whole outer opening portion does not overlap the inner opening portion;
making the pressure of the inside of the inner tube negative;
making a thrombus enter the inside of the inner tube through the outer opening portion and the inner opening portion by positioning the outer tube at the open position; and
shearing the thrombus which has entered the inside of the inner tube using both edge portions of the outer opening portion and the inner opening portion by relatively moving the outer tube to the closed position.

13. The method according to claim 12, comprising:
aspirating the thrombus through the inside of the inner tube.

14. The method according to claim 12, comprising:
configuring the outer tube so as to be rotatable around an axis of the inner tube.

15. The method according to claim 12, comprising:
configuring the outer tube so as to be slidable in an axial direction of the inner tube.

16. The method according to claim 12,
wherein the inner tube has a plurality of inner opening portions, and
wherein the outer tube has a plurality of outer opening portions respectively corresponding to the plurality of inner opening portions.
